(19) United States
(12) Patent Application Publication
(10) Pub. No.: US 2006/0015075 A1
(43) Pub. Date: Jan. 19, 2006

(54) Guarded infusor needle and infusor locking system

(75) Inventors: Ernesto E. Blanco, Belmont, MA (US); Gilbert B. Wilshire, Vienna, VA (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Erblan Surgical Inc., Belmont, MA (US)

(21) Appl. No.: 11/171,288

(22) Filed: Jul. 1, 2005

Related U.S. Application Data
(63) Continuation-in-part of application No. 10/792,821, filed on Mar. 5, 2004.
Application No. 10/792,821 is a continuation-in-part of application No. 10/324,050, filed on Dec. 20, 2002, now Pat. No. 6,716,201.
Application No. 10/324,050 is a continuation of application No. 09/598,453, filed on Jun. 22, 2000, now Pat. No. 6,497,687.
(60) Provisional application No. 60/586,295, filed on Jul. 9, 2004.
(60) Provisional application No. 60/586,294, filed on Jul. 9, 2004.
(60) Provisional application No. 60/140,409, filed on Jun. 22, 1999.
(60) Provisional application No. 60/452,040, filed on Mar. 6, 2003.
(60) Provisional application No. 60/494,122, filed on Aug. 12, 2003.

(51) Int. Cl. A61M 25/00

(52) U.S. Cl. 604/264

(57) ABSTRACT

A surgical device including a penetrator configured to pierce a membrane of a patient and a guard positionable to permit and to prohibit the penetrator from piercing the membrane. The guard includes a shield member movable along a longitudinal axis between a first position covering the penetrator and a second position uncovering the penetrator, the shield member including a stop member; a locking member configured to contact the stop member to prohibit the shield member from moving from the first position to the second position; and an unlocking member surrounding a portion of the shield member and configured to move along the longitudinal axis to move the locking member out of contact with the stop member to permit the shield member to move from the first position to the second position.

### CROSS REFERENCE TO RELATED APPLICATIONS

This application claims the benefit of the earlier filing date of U.S. Provisional application Nos. 60/586,294, filed Jul. 9, 2004, entitled “INFUSOR LOCKING SYSTEM,” and 60/586,295, filed Jul. 9, 2004, entitled “GUARDED NEEDLE OR PENETRATOR,” the entirety of each of which is incorporated by reference herein. The foregoing invention is related in terms of structure and functioning and also claims the benefit of U.S. application Ser. No. 10/792,821, filed Mar. 5, 2004, which claims priority to U.S. Provisional application Nos. 60/452,040, filed Mar. 6, 2003 and 60/494,122 filed Aug. 12, 2003, and U.S. application Ser. No. 10/324,050 filed Dec. 20, 2002, which is a Continuation application of Ser. No. 09/598,453, filed Jun. 22, 2000 and claims priority to U.S. Provisional application No. 60/140,409, filed Jun. 22, 1999, the disclosure of each of which is herein incorporated by reference.

### BACKGROUND OF THE INVENTION

1. FIELD OF THE INVENTION

The present invention is directed to a guarded infusor needle and infusor locking system which can be utilized with an insufflation needle, a Veress needle, a trocar or other type of penetrator used in surgery or other fields and industries. Particularly, this locking system is adaptable for any other type of use outside the field of surgery requiring a guarded penetration of an item with a blade.

2. DESCRIPTION OF THE RELATED ART

Conventional infusion needles are unsafe on at least four counts. First, besides penetrating too deeply with an exposed cutting tip, such needles have no flow-through safety fluid communication feature for inducing a potential space beneath the peritoneum. In addition, they have no safety locking systems and have the persistent problem of fully exposing the most critical axially movable fluid tube fixtures thereof which hang from the proximal end and are connectable to a catheter. Such connection must never be subjected to external interference or be accidentally struck by operators who may thus otherwise impede safe use of the needle.

Accordingly, an infusor, needle, and/or penetrator addressing these safety concerns is desired.

### SUMMARY OF THE INVENTION

One aspect of the present invention is a surgical device including a penetrator configured to pierce a membrane of a patient and a guard positionable to permit and to selectively prohibit the penetrator from piercing the membrane. The term penetrator is used to generally include any penetration needle or other device and may include an infusor capable of performing fluid injection at the moment of penetration as well as prior to and subsequent to penetration. The guard includes a shield, guard or other protective cover member movable along a longitudinal axis between a first position covering the penetrator and a second position uncovering the penetrator, the shield member also including, if desired, a stop; a locking member configured to contact the stop to prohibit the shield member from moving from the first position to the second position; and an unlocking member surrounding a portion of the shield member and configured to move along the longitudinal axis to move the locking member out of contact with the stop to permit the shield member to move from the first position to the second position.

Another aspect of the present invention is a surgical device including a penetrator configured to pierce a membrane of a patient, the penetrator comprising an elongated needle member having a blade mounted thereto and having a first blade edge and, if desired, a second blade edge, forming a first blade angle therebetween; and a guard mounted on a second elongated member and positionable within the first elongated member, the guard including at least one shield member having first and second edges forming a second angle therebetween and which is movable along a longitudinal axis of the first elongated member between a first position covering the first and second blade edges and a second position uncovering the first and second blade edges. The first angle formed between the first and second blade edges is greater than the second angle between the first and second edges of the shield member.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a perspective view of a preferred embodiment of the present invention;

FIG. 2 is a side view thereof;

FIG. 3 is a top view thereof;

FIG. 4 is a side cross-sectional view thereof in the locked position prior to arming;

FIG. 5 is a side cross-sectional view thereof in the armed position;

FIG. 6 is a side cross-sectional view thereof during the penetration phase;

FIG. 7 is a perspective view of the guards with the guards protecting the blade;

FIG. 8 is a side view of the guards when protecting the blade;

FIG. 9 is a top view of the guards showing the guards protecting the blade;

FIG. 10 is a front view of the guards;

FIG. 11 is a cross-sectional view of the guards taken from a proximal side of the guards;

FIG. 12 is a perspective view of the guards with the blade exposed;

FIG. 13 is a side cross-sectional view of an alternative embodiment of the present invention;

FIG. 14 is a top view of the guards of the alternative embodiment of the present invention; and

FIG. 15 is a side view of the guards of the alternative embodiment of the present invention.

### DETAILED DESCRIPTION OF THE INVENTION

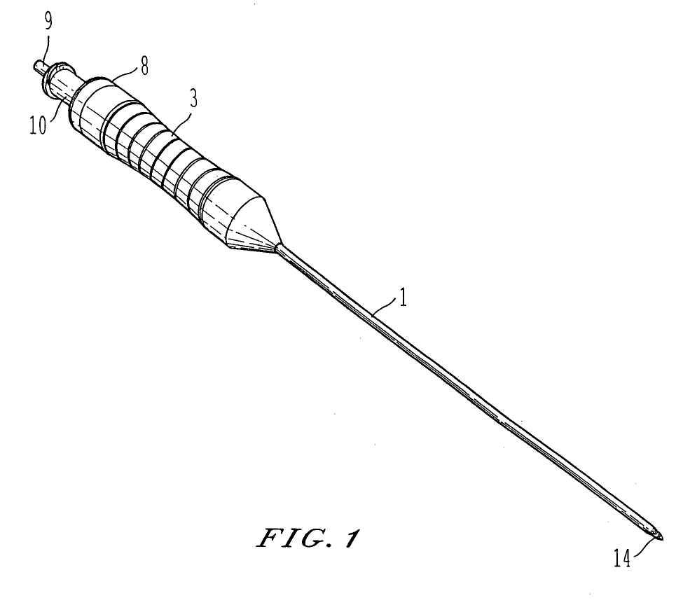

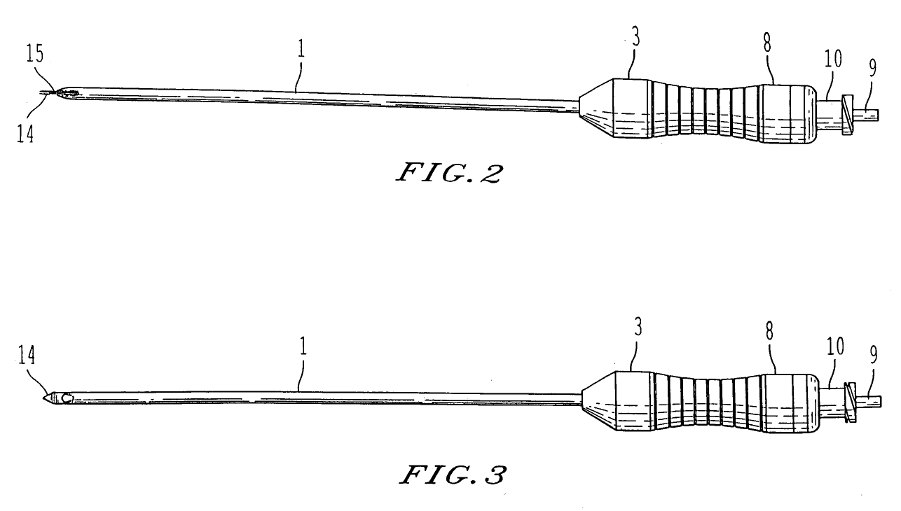

Referring now to the drawings, wherein like reference numerals designate identical or corresponding parts throughout the several views, a preferred embodiment is shown in FIGS. 1-12 and an alternative embodiment in FIGS. 13-15.

In one embodiment, the present invention provides a coaxial safety locking system for an infusion or insufflation type needle which permits penetration of the peritoneum (or any layer of tissue or membrane) of an individual in a safe manner due to the projecting forward of the guard mechanism prior to complete penetration of the peritoneum by the blade of the needle. The safety lock mechanism of the present invention permits reactivation of the guard mechanism for penetration sequentially through more than one layer of tissues or membranes by the blade of the infusor of an individual or when used in other types of surgery not involving the peritoneum. The infusor may comprise an infusion or insufflation needle, a trocar or any type of penetrator and may or may not permit infusion of a fluid, typically a gas, through the needle and thus permit a puff of such fluid towards the internal tissue upon initial penetration of the peritoneum or other tissue layer. This helps overcome the inherent problems with conventional infusion needles.

In another embodiment, the present invention provides an infusion or insufflation type needle without the use of a safety locking system for the guard that can be reactivated but which permits penetration of the peritoneum of an individual in a safe manner due to the projecting forward of a guard mechanism prior to complete penetration of the peritoneum by the blade of the needle. The present invention permits penetration sequentially through at least one layer of the peritoneum or other tissue of an individual by the blade of the infusor or when used in other types of surgery not involving the peritoneum. The infusor may comprise an infusion or insufflation needle, a trocar or any type of penetrator and may or may not permit infusion of a fluid, typically a gas, through the needle and thus permit a puff of such fluid to be communicated towards the internal tissue upon initial penetration of the peritoneum or other tissue layer. Such communication can be initiated of course before, during and/or after penetration begins, if desired.

The above-noted difficulties are overcome by the present invention which provides for flow through of the safety fluid to induce a potential space, provide a safe and reliable locking mechanism and permit in a safe and convenient manner communication of the fluid to the infusor needle.

One characteristic of the present invention is that such has an arming safety feature. This permits selective rearming of the needle so as to permit it to be rearmed prior to penetration through each of multiple layers of the tissues of an individual or of an object if used in a field other than surgery.

Another characteristic of the present invention is that such has a flow through feature of the safety fluid between the guards of the guard mechanism and the blade. This avoids the need for an aperture being provided the guards for passage of the safety fluid.

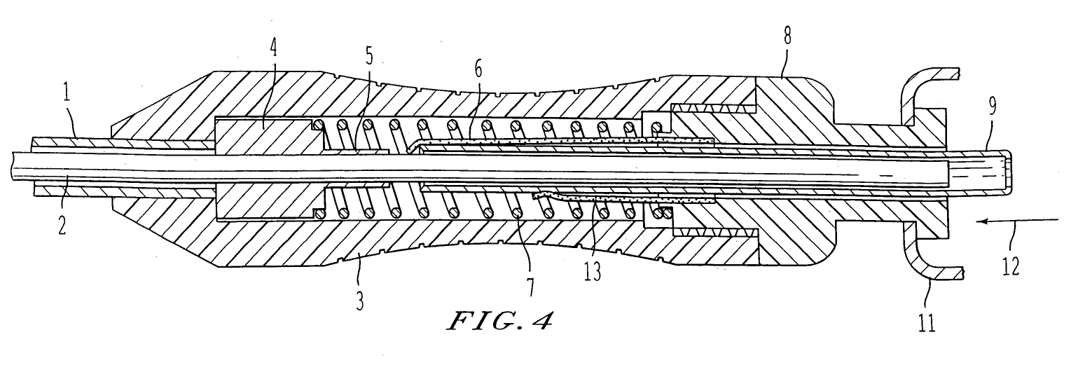

In view of the foregoing, the present invention, as illustrated in FIGS. 1-4 disclosing details of the present invention, includes a penetrator tube or cannula (1), a guards tube or obturator (2), a handle (3), a push disc (4), a locking tube (5), a locking jaw (6), a coiled spring (7), a handle cap (8) which may be threaded if desired, an arming tube (9), a Luer (10), a feeding catheter (11), and a friction latch (13) wherein the arming tube (9) is pushed by an operator in the direction of arrow (12) for rearming of the guards. FIG. 4 discloses the details of the present invention and sets forth a longitudinal cross-section of the handle when in the locked position prior to arming. In this figure, the locking jaw (6) is shown resting on the guards tube (2), thus impeding the motion of the locking tube (5) and therefore locking the system from motion.

As shown in FIG. 5, arming of the system is done by pushing the arming tube (9) in the direction of arrow (12) and forcing the locking jaw (6) upwards and out of the way of the locking tube (5). Any force applied to the locking tube (5) by the guards tube (2) to which it is attached will allow the guards tube (5) to move to the right under the locking jaw (6) without interference, therefore allowing retraction of the tip guards (14) and permitting exposure of a cutting blade (15) attached to the penetrator (1). While the preferred embodiment utilizes a metal blade, it is understood that any sharpened, substantially flat member made of another material such as plastic or a composite material could be utilized. The system is now ready for penetration across the tissue when pushed against them and thus is now said to be armed.

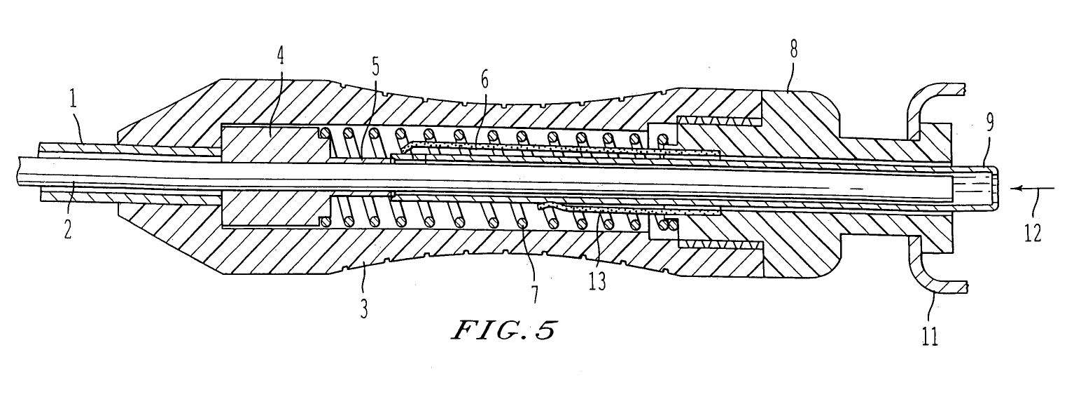

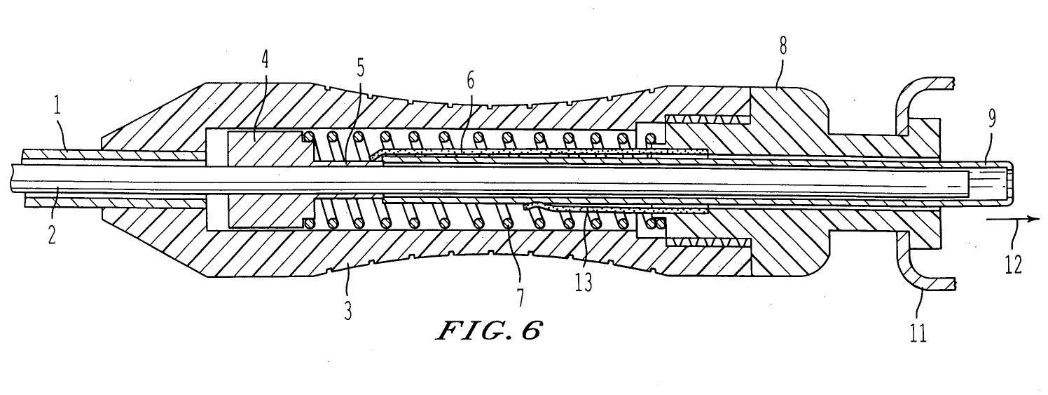

FIG. 6 illustrates the penetration phase wherein the guards tube (2) attached to the push disc (4) and its locking tube (5) is now able to move across to the right in such figure and push the arming tube (9) out of the way, thus overcoming the holding friction of the friction latch (13) underneath the arming tube (9).

As shown in FIG. 4 immediately after the penetration is finished, the guards tube (2) and the locking tube (5) return to the left under the action of the spring (7) and the locking jaw (6) drops from being locked over the locking tube (5) and on top of the guards tube (2) while the arming tube (9) remains held by the friction latch (13). This comprises a return to the locking position. Any force applied to the guards at the left end and to the guards tube (2) will be opposed by the front contact between the tip of the locking jaws (6) and the face of the locking tube (5).

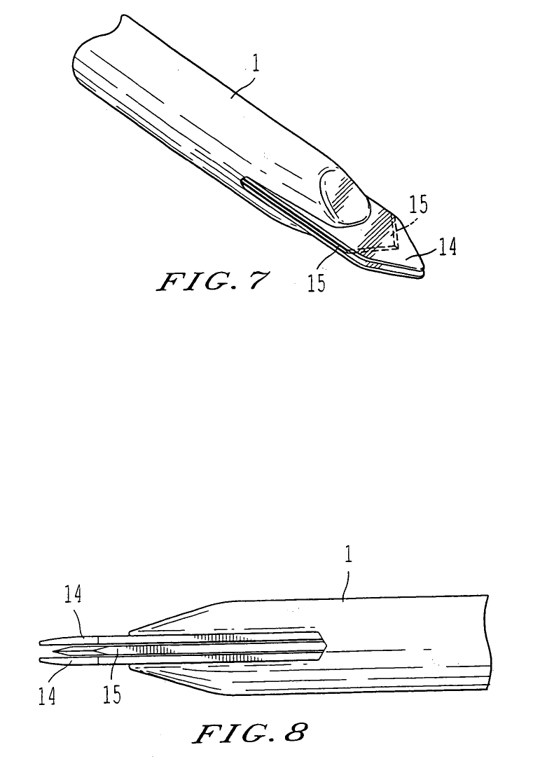

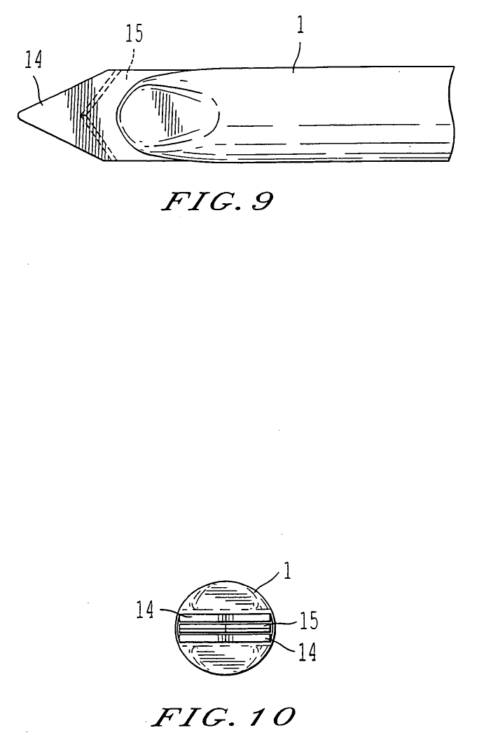

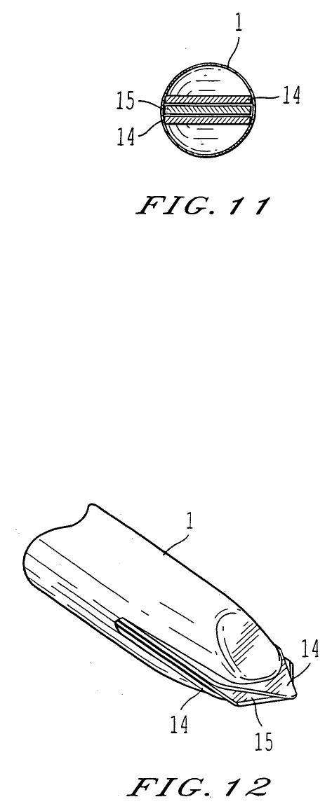

The distal end of the guards tube (2) is formed by a pair of guards (14, 14) which are parallel with and in close proximity with blade (15), as illustrated in the figures. The guards are connected to the tube (2) in a spaced apart manner near the blade (15) so as to guard opposite flat sides of the blade when in the guarded position. This can be seen, for example, in FIG. 12 wherein the guards have been retracted to permit cutting by the cutting blade 15 and the apex angle of the tip portion of the guards (14) is less than that of the tip portion of the blade (15). In the figures, FIG. 7 shows the guards in a position when protecting the blade (15), FIG. 8 shows a side view of FIG. 7, FIG. 11 shows a cross-sectional view in a direction towards the distal end of the guards and FIG. 12 shows the blade and guards when the blade has been exposed for cutting.

A second embodiment of the present invention is shown in FIGS. 13-15. This embodiment includes a penetrator tube or cannula (1), a guards tube or obturator (2), a handle (3), a disc (4) fixedly secured to the guards tube (2), a coiled spring (7), a cavity (20) formed in the body (3) within which the spring is housed, a threaded handle cap (8) and a Luer (10) having a passageway (22) for the safety fluid passageway, wherein the disc (4) and guards tube or obturator (2) are biased in the direction of the blade (15) for guarding a blade by a pair of guards (14, 14) fixed to the distal end of the guards tube (2). FIG. 13 discloses the details of the present embodiment and sets forth a longitudinal cross-section of the handle (3) shown in the state when biasing the disc (4), the guards tube (2) and the guards (14, 14) towards the blade (15).

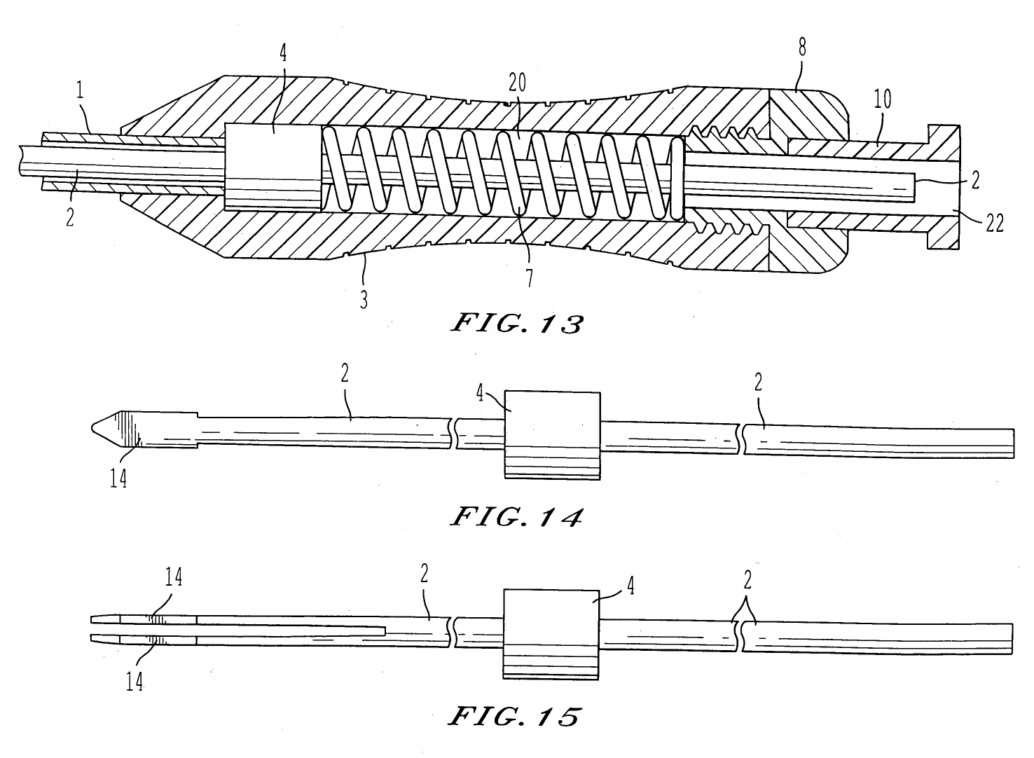

FIGS. 14 and 15 illustrate the structure of the guards (14, 14) which are biased towards the blade (15) to protect and cover such blade, as shown in a top plan view and side elevational view, respectively.

As shown in FIG. 13, the guards tube or obturator (2) and the guards (14, 14) are movable to the right to compress the spring (7) when the guards are engaged with the peritoneum or other tissue of an individual. Any force applied to the guards (14, 14) at the left end of the guards tube (2) will be opposed by the bias the spring (7) in the direction toward blade (15). As in the previous embodiment, the distal end of the guards tube (2) is formed by a pair of guards (14, 14) which are substantially parallel with and in close proximity with blade (15).

Obviously, numerous modifications and variations of the present invention are possible in light of the above teachings. It is therefore to be understood that within the scope of the appended claims, the invention may be practiced otherwise than as specifically described herein.

1. A surgical device, comprising:
a penetrator configured to pierce a membrane of a patient; and
a guard positionable to selectively permit and prohibit guarding of the penetrator from piercing the membrane, the guard comprising:
a shield member movable along a longitudinal axis between a first position covering the penetrator and a second position uncovering the penetrator, the shield member comprising a stop member;
a locking member configured to contact the stop member to prohibit the shield member from moving from the first position to the second position; and
an unlocking member surrounding a portion of the shield member and configured to move along the longitudinal axis to move the locking member out of contact with the stop member to permit the shield member to move from the first position to the second position.

2. The surgical device according to claim 1, wherein the penetrator comprises a needle and the membrane comprises one of a peritoneum, a tissue layer and a membrane layer of a patient.

3. The surgical device according to claim 2, wherein the needle comprises an infusion needle.

4. The surgical device according to claim 2, wherein the needle comprises an insufflation needle.

5. The surgical device according to claim 1, wherein the penetrator comprises one of a trocar and a needle.

6. The surgical device according to claim 1, wherein the shield member comprises at least one of a protective member, a shield member and a guard connected to a guard rod, the protective member being movable to cover and to uncover the penetrator, and
the unlocking member comprises an unlocking rod surrounding a portion of the guard rod, the unlocking rod is configured to be moved along the longitudinal axis to permit the shield member to move from the first position to the second position.

7. The surgical device according to claim 6, wherein the unlocking rod is configured to be moved along the longitudinal axis between an armed position in which the locking member is moved out of contact with the stop member and an unarmed position in which the locking member is in contact with the stop member.

8. The surgical device according to claim 7, wherein the guard further comprises a friction member configured to apply a frictional force to the unlocking rod.

9. The surgical device according to claim 6, wherein the stop member comprises a protrusion extending from the guard rod.

10. The surgical device according to claim 9, wherein the protrusion comprises a stepped protrusion.

11. The surgical device according to claim 10, wherein the guard further comprises a biasing member contacting a portion of the stepped protrusion to urge the shield member toward the first position.

12. The surgical device according to claim 11, wherein the guard further comprises a frictional member configured to apply a frictional force to the unlocking rod.

13. A surgical device, comprising:
a tube comprising a penetrator and a guard positionable to permit and prohibit the penetrator from piercing a peritoneum of a patient;
a handle connected to the tube, the handle defining a void;
a guard tube disposed in the void and connected to the guard such that the guard and guard tube are selectively movable along a longitudinal axis from a covering position in which the guard covers the penetrator to an uncovering position in which the guard uncovers the penetrator;
a locking tube disposed on the guard tube;
a locking member disposed in the void and movable between a locked position in which the locking member contacts the locking tube to prevent the guard from uncovering the penetrator and an unlocked position in which the locking member is out of contact with the locking tube to permit the guard to uncover the penetrator; and
an unlocking member surrounding the guard tube and movable along the longitudinal axis between an unarmed position in which the unlocking member permits the locking member to contact the locking tube to prevent the guard from uncovering the penetrator and an armed position in which the unlocking member moves the locking member out of contact with the locking tube to permit the guard to uncover the penetrator.

14. The surgical device according to claim 13, wherein the penetrator comprises one of a needle and a trocar.

15. The surgical device according to claim 14, wherein the needle comprises one of an infusion needle and an insufflation needle.

16. The surgical device according to claim 13, further comprising:
a biasing member disposed in the void, the biasing member being configured to apply a biasing force to urge the locking tube, guard tube, and guard from the uncovering position to the covering position.

17. The surgical device according to claim 16, further comprising:
a friction latch configured to apply a frictional force to the unlocking member.

18. The surgical device according to claim 17, wherein the unlocking member extends from an end of the handle opposite an end to which the tube is connected.

19. The surgical device according to claim 18, wherein the unlocking member comprises an end portion configured to be manually urged from the unarmed position to the armed position.

20. A method of using a surgical device including a penetrator configured to pierce a membrane of a patient and a guard positionable to permit and to prohibit the penetrator from piercing the membrane, the guard including a shield member movable along a longitudinal axis between a first position covering the penetrator and a second position uncovering the penetrator, the shield member including a stop member, the guard including a locking member configured to contact the stop member to prohibit the shield member from moving from the first position to the second position, and the guard including an unlocking member surrounding a portion of the shield member and configured to move along the longitudinal axis to move the locking member out of contact with the stop member to permit the shield member to move from the first position to the second position, the method comprising:
moving the unlocking member along the longitudinal axis to uncover the penetrator; and
piercing the membrane with the uncovered penetrator.

21. The method according to claim 21, wherein piercing the membrane comprises piercing the membrane with one of a needle and a trocar.

22. A method of using a surgical device, comprising:
moving an unlocking device in a longitudinal direction such that the unlocking device moves a locking device out of contact with a stop member disposed on a shield member; and
moving the shield member along the longitudinal axis to uncover a penetrator to pierce a membrane of a patient.

23. The method according to claim 22, wherein moving the unlocking device comprises moving the unlocking device to move the locking device in a direction substantially perpendicular to the longitudinal direction.

24. The method according to claim 22, further comprising:
piercing the membrane of the patient with the penetrator; and
covering the penetrator with the shield member.

25. The method according to claim 24, wherein covering the penetrator comprises applying a biasing force to the stop member to move the shield member to cover the penetrator.

26. A surgical device, comprising:
a penetrator configured to pierce a membrane of a patient, the penetrator comprising an elongated needle member having a blade mounted thereto having a first blade edge and a second blade edge; and
a guard mounted on a second elongated member and positionable within the first elongated member, the guard comprising at least one shield member having first and second edges movable along a longitudinal axis of said first elongated member between a first position covering the first and second blade edges and a second position uncovering the first and second blade edges,
wherein a first angle formed between the first and second blade edges is greater than a second angle between said first and second edges of said shield member.

27. The surgical device according to claim 26, wherein the shield member further comprises a tube connected to the shield member, the tube having a hollow interior.

28. The surgical device according to claim 26, wherein the shield member further comprises a tube connected to the shield member, the tube having a hollow interior for permitting fluid flow through the guard.

29. The surgical device according to claim 26, wherein the shield member comprises at least one plate member.

30. The surgical device according to claim 26, wherein the shield member comprises a substantially planar first surface member offset from a substantially planar bottom surface member.

31. The surgical device according to claim 26, further comprising:
a second shield member movable along the longitudinal axis between the first and second positions.

32. The surgical device according to claim 31, wherein at least one of the shield members comprises a substantially flat top surface offset from a substantially flat bottom surface.

33. The surgical device according to claim 31, wherein one of the shield members is disposed adjacent a top surface of the penetrator, and the other one of the shield members is disposed adjacent a bottom surface of the penetrator which is opposite the top surface.

34. The surgical device according to claim 33, wherein the second shield member comprises first and second edges, with a third angle between the first and second edges of said second shield member, the third angle being greater than the second angle.

35. The surgical device according to claim 34, wherein the first and third angles are substantially equal.

36. The surgical device according to claim 26, wherein the guard further comprises a hollow tube including a stop member, the tube being connected to and configured to move with the shield member between the first and second positions.

37. The surgical device according to claim 36, wherein the guard further comprises a biasing member configured to urge the tube and the shield member from the second position to the first position.

38. The surgical device according to claim 37, wherein the biasing member comprises a spring.

39. A surgical device, comprising:
a first tube;
a handle connected to the first tube;
a penetrator connected to an end of the first tube, the penetrator comprising a left blade edge and a right blade edge configured to pierce a membrane of a patient;
a second tube disposed in an interior of the tube and in an interior of the handle; and
a shield member connected to an end of the second tube and disposed adjacent the penetrator in the first tube, the shield member comprising a left covering edge and a right covering edge, the shield member being configured to move along a longitudinal axis between a first position covering the penetrator and a second position uncovering the penetrator,
wherein an angle between the left and right blade edges is greater than an angle between the left and right covering edges.

40. The surgical device according to claim 39, further comprising:
a biasing member configured to urge the shield member from the second position to the first position.

41. The surgical device according to claim 40, wherein the rod comprises a stopper, and the biasing member is disposed in the interior of the handle in contact with the stopper.

42. The surgical device according to claim 39, further comprising:
a second shield member connected to an end of the second tube and disposed adjacent the penetrator, the second shield member comprising second left and right covering edges, the second shield member configured to move along the longitudinal axis between the first and second positions,
wherein the shield members are disposed on opposite sides of the penetrator.

43. The surgical device according to claim 42, wherein the shield members comprise substantially flat plates.

44. A method of using a surgical device including a first tube, a handle connected to the first tube, a penetrator connected to an end of the first tube, the penetrator including left and right blade edges configured to pierce a membrane of a patient, a second tube disposed in an interior of the first tube and in an interior of the handle, and a shield member connected to an end of the second tube and disposed adjacent the penetrator, the shield member including left and right covering edges, the shield member being configured to move along a longitudinal axis between a first position covering the penetrator and a second position uncovering the penetrator, where a first angle between the left and right blade edges is greater than an angle between the left and right covering edges, the method comprising:
uncovering the penetrator; and
piercing the membrane with the penetrator.

45. The method according to claim 44, further comprising:
covering the penetrator with the shield member after removal of the surgical device from the membrane.

46. A method of using a surgical device, comprising:
piercing a membrane of a patient with a penetrator blade comprising a left blade edge and a right blade edge; and
moving a first shield member comprising a left cover edge and a right cover edge to cover the penetrator,
wherein an angle between the left and right blade edges is greater than an angle between the left and right cover edges.

47. The method according to claim 46, further comprising:
moving the first shield member to uncover the penetrator before piercing the membrane of the patient.

48. The method according to claim 47, further comprising:
disposing the first shield member and a second shield member on opposite sides of the penetrator, the shield members comprising substantially flat plates.
